(19) United States
(12) Patent Application Publication
(10) Pub. No.: US 2013/0053723 A1
(43) Pub. Date: Feb. 28, 2013

(54) Pleural Pressure Indicator

(71) Applicants: Andre Marcel Leveque, Magnolia, TX (US); Jacob Gregory Vonderharr, Vancouver, WA (US)

(72) Inventors: Andre Marcel Leveque, Magnolia, TX (US); Jacob Gregory Vonderharr, Vancouver, WA (US)

(73) Assignee: ISG Inc., Magnolia, TX (US)

(21) Appl. No.: 13/596,967

(22) Filed: Aug. 28, 2012

Related U.S. Application Data
(60) Provisional application No. 61/529,443, filed on Aug. 31, 2011.

(51) Int. Cl. A61B 5/03

(52) U.S. Cl. 600/561

(57) ABSTRACT

The pleural pressure indicator is a diagnostic device for use in the field where more sophisticated electronics are not permissible, though early detection of pneumothoraces relative to penetrating chest trauma is paramount to survival.

When the device is used with the placement of a needle thoracentesis, it serves the user by indicating flow to and from the chest cavity. This indication is supportive of the user's diagnosis of the patients' respiratory condition.

When a patient is suffering from a tension pneumothorax, positive air flow will be indicated by the device. The user can switch the check valve in the device flow path to manage intrathoracic pressure relief, therapeutically reducing the tension pneumothorax, increasing survivability.

The pleural pressure indicator is a compact, portable, handheld device that is simple to operate, compatible with needles, catheters, and syringes and simple to read for a quick indication of a patient's condition.

### BACKGROUND OF THE INVENTION

The pleural pressure indicator is a handheld diagnostic device for use in the field where more sophisticated electronic devices are not available. Utilized by a trained individual, the pleural pressure indicator is a diagnostic and therapeutic tool able to support the field diagnosis of a tension pneumothorax.

The tension pneumothorax is a life threatening condition that results from the accumulation of air within the pleural cavity, subsequent to chest injury from penetrating trauma. As the casualty breathes, air escapes from the injury site at the lung and is allowed to accumulate within the pleural cavity. Air accumulates with each breath the casualty takes and is unable to return through the lung tissue where the injury site serves as a one way valve. As accumulated air increases so does pressure within the chest wall which inhibits the patient's attempts to produce negative pressure during inhalation. This subsequent increase of intrathoracic pressure defines the development of the tension pneumothorax and leads to an increase in field mortality. Additionally, where accumulated air produces pressure sufficient enough to reduce cardia preload this results in circulatory compromise.

The successful detection of the tension pneumothorax by field personnel is an advanced skill and requires many hours of training. A device that can, within the field environment, determine the buildup of pressure would serve to be lifesaving by providing a reliable indication of the internal condition of the pleural space, where make-shift and impromptu techniques are currently being used. This device would be especially suited for the diagnosis and relief of patients in low light, dark conditions and other non-ideal environments, such as aboard aircraft.

The pleural pressure indicator can demonstrate the onset of a pleural pneumothoraces due to the nature of the flow sensor. The sensor is rated to detect air flow direction and velocities as low as 1 m/s which is necessary to detect the onset of a pneumothorax and subsequent tension pneumothorax. Where a patient is in a normal non-tensile condition the device will demonstrate neutral or negative air flow. In addition to the sensor, supporting circuitry will retrieve the signal and then report to the user an indication based on positive, negative or neutral air flow. This indication is demonstrated through the use of LEDs (Light Emitting Diode), an LCD (Liquid Crystal Display) screen or even sound.

With a sleek, compact, and simple design the device remains intuitive, presenting to the user a short learning curve to competency and practical confidence associated with the future diagnoses of patients in the field. The invention is easily packed in a personal medical aid kit and can be ready for use at any time.

The relevant prior art found is as follows: U.S. Pat. No. 4,164,938 describes a medical pressure gauge and indicator device that is intended to diagnose the presence of a tension pneumothorax by indication through the resulting expansion and contraction of a bulb integral to the device. The expansion takes place when pressure within the patient's pleural cavity is greater than atmospheric. U.S. Pat. Nos. 4,664,660 and 6,770,063 B2 describe devices used for draining fluids from body cavities, in specific example, the pleural cavity. The fluid is drained through a catheter into a connected reservoir. U.S. Pat. Nos. 4,447,235 and 5,997,486 describe devices used for clinical thoracentesis procedures in which body fluids are drained from a cavity into an outside reservoir device away from the patient.

### SUMMARY OF THE INVENTION

The pleural pressure indicator is a handheld diagnostic device for use in the field where more sophisticated electronic devices are not available. Utilized by a trained individual, the pleural pressure indicator is a diagnostic and therapeutic tool able to support the field diagnosis of a tension pneumothorax.

The current invention is intended to be a common article that should accompany medical care provider kits as well as the personal care kits carried by non-medical personnel. The device will supplement current methodologies that lack the means to identify the casualty's respiratory condition or advance those impromptu field techniques that remain inaccurate or where respiratory conditions remain ultimately undetectable in non-ideal environments.

The key component to the device is the use of a temperature dependent resistor based sensor for detecting air flow. The device bears the ability to demonstrate sensed air flow through the use of indicators which could include: LEDs (Light Emitting Diodes) an LCD (Liquid Crystal Display), or a sound emitter all of which would be oriented to demonstrate air flow and its relative magnitude in a linearly stepping sequence. A check valve is included to manage the release of the accumulated intrathoracic pressure and eliminate the possibility of an open pneumothorax. Remaining components provide power and signal reporting through a circuit. All components are contained in a sleek, pen-like housing that supports concurrent manipulation, indication, and reading of the device.

The pleural pressure indicator will provide a lightweight, compact, field-ready solution to medical users attempting to diagnose patients with symptoms of pneumothorax or subsequent tension pneumothorax. The object of the invention is to simplify the task of diagnosis, thereby eliminating the guess work and inaccuracies associated with impromptu techniques.

### BRIEF DESCRIPTION OF THE DRAWINGS

FIG. 1 is a cutaway side view of the conceptual current invention, showing an attached needle and a view of the primary containing components.

FIG. 2 is a schematic side view of the basic functioning of the flow sensor and it's sub-features.

FIG. 3 is a flow chart indicating the process that takes place from sensing to indicating in the current invention.

FIG. 4 is a diagram of a human patient's chest area displaying the proper location of a needle thoracentesis that is necessary to allow the current invention to detect the patient's condition with respect to a tension pneumothorax.

### DETAILED DESCRIPTION OF INVENTION

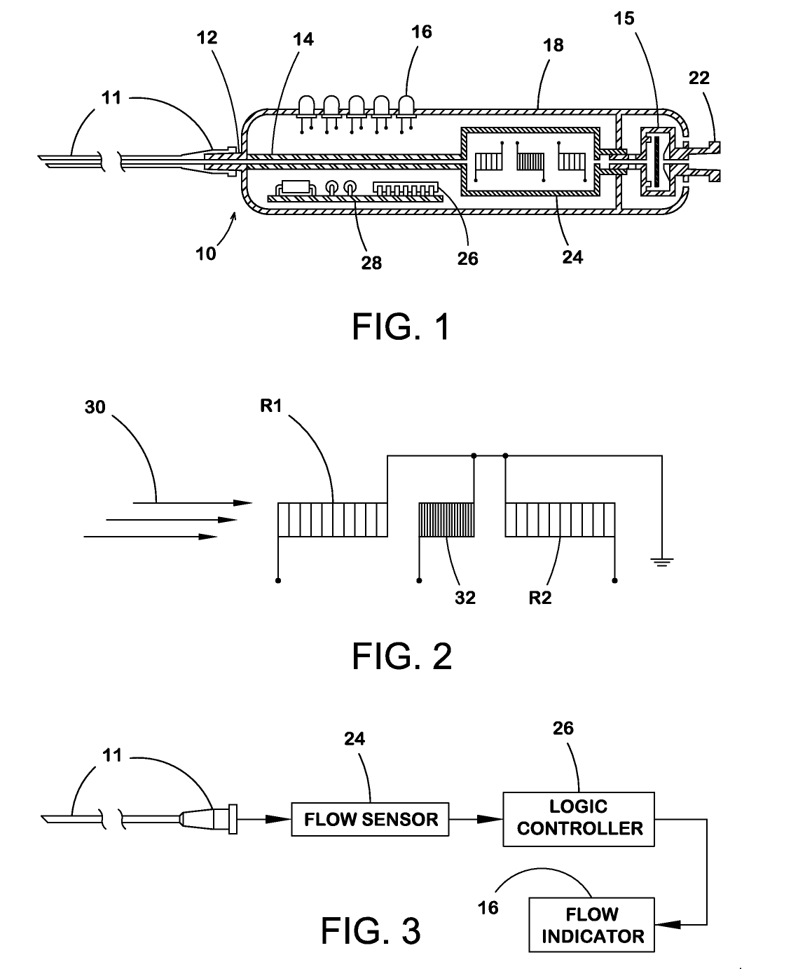

As shown in FIG. 1, the current invention 10 comprises of a handheld body 18 containing several features to allow proper operation and interface with the user. The features include inlet 12 and outlet 22 ports, user activated check valve 15, a temperature dependent resistor based sensor 24, software based logic controller 26, indicator 16, and a protective case 18 integrating the previous features. When the device is held in hand by the user, the proximal end with the integral inlet 12 is designed to mate with a medical catheter 11 for sensing positive pressure or lack thereof in a patient's pleural cavity through the proper use of a catheter over the medical needle 11 intercostally 44 by means of air flow 30.

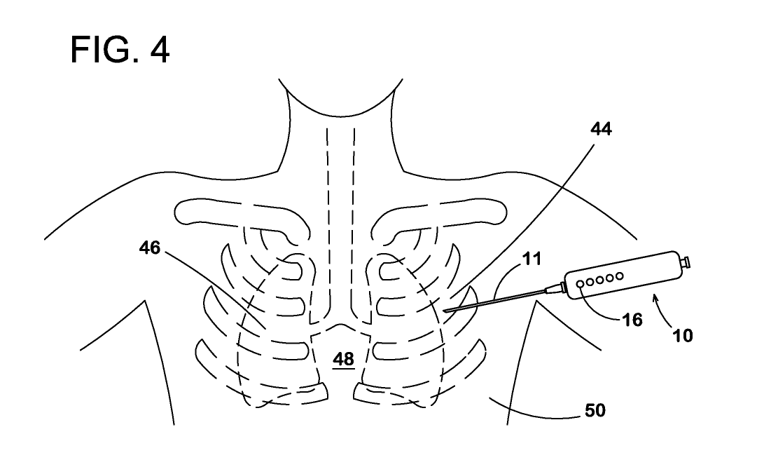

According to the inlet 12 feature, the inlet feature is included to interface the main body 18 of the invention containing the sensing element 24 to a catheter 11 that is properly located on the patient as shown in FIG. 4.

According to both the inlet 12 and outlet 22 features of the present invention, the inlet and outlet ports are designed to allow through air flow and to mate with medical supplies that utilize a 6% taper Luer fitting as required in ISO 594-1: 1986 for proper adaptation specifically the inlet to mate with catheter 11.

According to the user activated check valve feature 15, the user activated check valve is necessary for the user to be able to manage the direction of air flow 30 once a tension pneumothorax has been detected, and the patient needs to be therapeutically relieved of the built up pressure in the chest 50 thus allowing the reduction in pressure surrounding the lung 46 to reduce.

According to the temperature dependent resistor based sensor 24 feature, this sensor 24 is placed in line connected via tubing 14 to the inlet 12 and outlet 22 ports of the invention. As shown in detail in FIG. 2, the sensor 24 will sense the flow direction of air traveling 30 through the ports 12, 22 and the check valve 15, in both the forward and reverse direction. As shown in FIG. 2, this is possible by means of temperature difference between two resistors R1 and R2 placed parallel to the air flow 30 path. When the air flow 30 travels past the sensor 24, heat from a heater coil 32 located between the two resistors R1 and R2 is directed to one of the resistors R1 or R2, depending on the direction of air flow 30. The increase in temperature will change the affected resistor's value of resistance, ultimately creating a differential between the two resistors R1 and R2. This difference can be used as an electrical indicator of the direction of air flow 30.

According to the software based logic controller 26 feature, a typical micro-controller chip is used to receive the resistance differential signal from the temperature dependent resistor based sensor 24 feature and produce a logical decision output based on the input it receives. Examples include: when there is no air flow, both of the resistors, R1 and R2, in the sensor 24 will have the same value. In this case, an indicator 16 would report a no-flow condition. When there is airflow, both of the resistors R1 and R2 in the sensor 24 will have a different resistance. Depending on the direction of air, the indicator 16 will demonstrate airflow and magnitude in a stepped sequence such as for outward flow of positive air pressure relieving past the chest wall 48, and for inward flow, or a vacuum, from normal breathing. The logical process is shown in a flow chart form in FIG. 3.

According to the visual indicator 16 features, an example is LEDs 16 that will illuminate given the electrical signal from the software based logic controller 26. The LEDs 16 will illuminate respective to the output given by the logic controller 26. Other, more advanced indicators such as a LCD reporting conditions by wording or symbols is possible. An audio indicator could also be included if a sound reporting the condition of the patient was required.

According to the protective case 18 feature, this feature is required to integrate the previous features into a handheld package that is protected from damage and isolated operation.

Remaining features are those that are associated with typical portable electrical operation to allow the functioning of items including the temperature dependent resistor based sensor 24, software based logic controller 26, and visual indicator 16. Typical features include but are not limited to: a battery, power switch, resistors, and a printed circuit board represented in whole by 28.

1. A diagnostic medical indicator device for the diagnostics and relief of a tension pneumothorax comprising of:
a) A catheterization device combination comprising: a catheter; and a medical hypodermic needle.
b) A means to detect gas flow when attached to said catheter when catheter is properly placed in a patient's pleural space.
c) A logic controller in which signals are generated from said gas flow detection means.
d) A user interface operated by said logic controller to indicate to the user the detected conditions of the patient.
e) A check valve to limit the direction of flow within the device upon the detection of positive pressure gas flow from the patient's pleural cavity.
f) A protective case housing previously mentioned components less the catheterization combination.

2. The device defined in claim 1 wherein said means to detect gas flow is a temperature dependent resistor based sensor.

3. The device defined in claim 1 wherein said means to detect gas flow is a pressure transducer sensor(s).

4. The device defined in claim 1 wherein said logic controller is a microcontroller integrated circuit.

5. The device defined in claim 1 wherein said user interface is in a stepped sequence fashion.

6. The device defined in claim 1 wherein said user interface is based on light emitting diodes.

7. The device defined in claim 1 wherein said user interface is a liquid crystal display.

8. The device defined in claim 1 wherein said user interface is a sliding mechanism means to indicate positive or negative pressure within the patients pleural cavity.

9. The device defined in claim 1 wherein said user interface is a sound based emitter.

10. The device defined in claim 1 wherein said device is disposable after use.

11. The device defined in claim 1 wherein said catheterization device combination is attached by a removable means such as the use of a luer type connection.

12. The device defined in claim 1 wherein said catheterization device combination is integral to the remaining device body.
